SEQUENCE LISTING (1 sequence)

SEQ ID NO: 1 - protein, 127 aa, Homo sapiens [sequence omitted]

(19) United States
(12) Patent Application Publication
(10) Pub. No.: US 2022/0211667 A1
(43) Pub. Date: Jul. 7, 2022

(54) COMPOSITION FOR THE INTRAPERITONEAL TREATMENT OF SECONDARY BACTERIAL PERITONITIS WITH REDUCTION OF COMPLICATIONS

(71) Applicant: Reponex Pharmaceuticals A/S, Hørsholm (DK)

(72) Inventors: Lars Otto Uttenthal, Madrid (ES); Torsten Bjørn, Kvistgård (DK)

(21) Appl. No.: 17/594,681

(22) PCT Filed: Apr. 23, 2020
(86) PCT No.: PCT/EP2020/061355

§ 371 Date: Oct. 26, 2021

(30) Foreign Application Priority Data
Apr. 28, 2019 (DK) PA201970266

(51) Int. Cl. A61K 31/407; A61K 31/4164; A61K 31/665; A61K 38/19; A61P 31/04

(52) U.S. Cl. CPC A61K 31/407; A61K 31/4164; A61P 31/04; A61K 38/193; A61K 31/665

(57) ABSTRACT

The present invention provides compositions comprising granulocyte-macrophage colony-stimulating factor and antibiotic agents to treat, pre-emptively treat or prevent infectious peritonitis or intra-abdominal infection and reduce the occurrence of local peritonitis or intra-abdominal abscess resulting therefrom by the intraperitoneal administration of the compositions.

### FIELD OF INVENTION

The present invention provides a composition comprising granulocyte-macrophage colony-stimulating factor (GM-CSF) in combination with fosfomycin and metronidazole for the prophylaxis, pre-emptive treatment or treatment of peritonitis of bacterial origin by direct instillation of the composition into the peritoneal cavity at surgery. As such, it will be useful in the fields of abdominal surgery and gastroenterology.

### BACKGROUND OF INVENTION

Peritonitis is defined as inflammation of the peritoneum, the serosal membrane that lines the abdominal cavity and covers the organs within it. The peritoneum, which is normally sterile, reacts to various pathologic stimuli with a fairly uniform inflammatory response. Depending on the underlying pathology, the resulting peritonitis may be infectious or sterile in origin (Daley 2013).

Sterile or aseptic peritonitis may be caused by irritants such as foreign bodies, bile from a perforated gall bladder or a lacerated liver, gastric acid from a perforated ulcer or fluid from a ruptured ovarian cyst, or may result from genetically determined disorders such as polyserositis or familial Mediterranean fever and autoimmune diseases such as systemic lupus erythematosus.

Infectious peritonitis is caused by the entry of microorganisms into the abdominal cavity. It is conventionally classified into primary, secondary or tertiary peritonitis (see Holzheimer 2001).

Primary infectious peritonitis refers to spontaneous microbial invasion of the peritoneal cavity. It is often called spontaneous bacterial peritonitis. This mainly occurs in infancy and early childhood, in cirrhotic patients with ascites and in immunocompromised patients.

Secondary infectious peritonitis, usually bacterial, refers to peritoneal infections secondary to intra-abdominal lesions, such as perforation of the hollow viscus, bowel necrosis, penetrating infectious processes or bacterial infection consequential to an originally aseptic peritonitis.

Peritoneal dialysis-associated peritonitis is a regrettably common, special type of secondary infectious peritonitis resulting from bacterial contamination introduced by peritoneal dialysis, an increasingly widespread treatment for end-stage renal failure.

Tertiary peritonitis is a less well-defined entity characterized by persistent or recurrent infections with organisms of low intrinsic virulence or with predisposition for the immunocompromised patient. It usually follows operative attempts to treat secondary peritonitis and is almost exclusively associated with a systemic inflammatory response.

Infectious peritonitis is often used synonymously with intra-abdominal infection or sepsis. In assessing the significance of the presence of microorganisms in the peritoneal cavity, it may be useful to distinguish between contamination (the presence of bacteria in normal sterile tissue without any host reaction), infection (the presence of bacteria in normal sterile tissue with a local inflammatory response), and sepsis (the systemic response to local infection).

A small proportion of cases of infectious peritonitis (1-5%) are caused by fungi, most commonly by Candida albicans, but also by other fungi in rare instances.

Clinically, peritonitis is often classified as either as local or diffuse. Local peritonitis refers to a site of infection, usually walled off or contained by adjacent organs, and may also be called an intra-abdominal abscess. Diffuse peritonitis is synonymous with generalized peritonitis that spreads to the entire abdominal cavity.

The incidence of aseptic peritonitis, with its disparate causes, has not been globally assessed. The incidence of non-sporadic causes relates to the prevalence of familial Mediterranean fever and to a fraction of patients with systemic lupus erythematosus. Global figures are also lacking for primary infectious peritonitis, but this has been estimated to affect 10-30% of all patients admitted to hospital with ascites (Wiest et al 2012). The incidence of secondary peritonitis is similarly difficult to assess. Intra-abdominal infections occur in 25% of patients with multiple organ failure in surgical ICU. Peritonitis was present in 8% of all cases in a large necropsy series. Peritoneal dialysis-associated peritonitis occurs in 50-60% of patients within the first year of dialysis and recurrent episodes are common. Overall, it can be estimated that cases of secondary peritonitis, constituting by far the largest proportion of all peritonitis cases, must amount to several hundred thousand patients per year in the USA and Europe.

### Granulocyte-Macrophage Colony-Stimulating Factor (GM-CSF) in Peritonitis

GM-CSF is a cytokine of the colony-stimulating factor family which is secreted by macrophages, T cells, mast cells, natural killer (NK) cells, endothelial cells and fibroblasts. It is also secreted by the peritoneal mesothelial cells, the main fixed cell component of the peritoneal membrane. Secretion from these cells may be spontaneous in culture or upregulated by Interleukin (IL)-1 (Lanfrancone et al 1992), or induced by epidermal growth factor (EGF) and tumor necrosis factor (TNF) (Demetri et al 1989). GM-CSF functions as a white blood cell growth factor, stimulating stem cells to produce granulocytes (neutrophils, eosinophils, and basophils) and monocytes. Monocytes exit the circulation and migrate into tissue, whereupon they mature into macrophages and dendritic cells. GM-CSF stimulates the proliferation and maturation of macrophages into dendritic cells, which orchestrate the responses of the surrounding neutrophils and T lymphocytes to local infectious or inflammatory processes.

In severe infections such as bacterial peritonitis, the normal immuno-inflammatory defense mechanisms involving macrophages, neutrophils and lymphocytes may not always function optimally for the patient's recovery. Volk et al (1991) described the loss of HLA-class II antigen expression and other phenotypical abnormalities of monocytes from patients with septic peritonitis and fatal outcome. These abnormalities were associated with functional defects of antigen presentation, formation of reactive oxygen species and cytokine secretion. This phenomenon was termed “immunoparalysis”, and its leading feature, the loss of HLA-DR antigen expression to <20%, was reversed by GM-CSF and interferon-gamma in vitro.

However, attempting to restore monocyte/macrophage function in bacterial peritonitis by giving systemic GM-CSF has given rise to contradictory results. Toda et al (1994) treated rats with peritonitis induced by cecal ligation and puncture with recombinant murine GM-CSF. The survival rate did not improve and animals died earlier than in the control group. Systemic GM-CSF inhibited early leukocyte sequestration in the peritoneal cavity. It was concluded that “care should be taken” in the clinical use of GM-CSF in severe infection. Similarly, Barsig et al (1996) found that prophylactic administration (by an unstated route) of murine GM-CSF neither augmented leukocyte numbers nor protected mice in a sub-lethal model of fecal peritonitis.

In contrast, Gennari et al (1994) found that subcutaneous injection of GM-CSF significantly reduced the mortality of mice that had been immunosuppressed by allogenic transfusion and subjected to cecal ligation and puncture. Macrophage and leukocyte numbers and function were not recorded. Austin et al (1995) demonstrated a prophylactic effect of intraperitoneal GM-CSF given to mice for 5 days after a traumatic injury and before inducing bacterial peritonitis by cecal ligation and puncture. This procedure improved survival, increased the yield of harvested peritoneal cells, improved aspects of peritoneal macrophage function and reduced bacterial growth indices.

In human patients with non-traumatic generalized abdominal sepsis treated with systemic antibiotics, subcutaneously administered GM-CSF reduced the rate of infectious complications and length of hospitalization (Orozco et al 2006).

Selgas et al (1996) tested the effects of intraperitoneally administered GM-CSF on the number and activation of peritoneal macrophages in peritoneal dialysis patients. There was a large increase in peritoneal macrophage numbers returning to baseline seven days after cessation of treatment. GM-CSF increased macrophage expression of CD11b/CD18 (CR3) and its counter-receptor CD54, indicating progression to a more activated state. Both the number of phagocytic cells and the phagocytic index were augmented. Peritoneal effluent cytokine-chemokine levels demonstrated an increase in IL-6 and MCP-1 levels, while TNF-alpha, IL-1, IL-8, MIP-1 alpha and RANTES were not significantly altered. GM-CSF administration did not affect the peritoneal transport of water or solutes. Minor flu-like symptoms were experienced by 2 of 8 patients (those showing the highest rise in cell numbers) on the third and last day of treatment. It was concluded that GM-CSF causes a marked and transient recruitment of primed macrophages into the peritoneum without inducing inflammatory parameters and might thus improve the peritoneal defensive capacity through potentiation of the effector functions of resident and newly recruited macrophages.

In a follow-up study, Schafer et al (1998) determined that the peritoneal macrophages were the likely source of the chemokines released upon intraperitoneal administration of GM-CSF.

### Antimicrobial Treatment of Bacterial Peritonitis

Treatment of infectious peritonitis involves correction of the underlying process, such as leakage of bacteria from a bowel perforation, administration of systemic antibiotics, and supportive therapy to prevent or limit secondary complications due to organ failure (Daley 2013). Early control of the septic source is mandatory and can be achieved operatively or non-operatively.

Antimicrobial/antibiotic treatment is regarded as essential and may be applied before, during and after any surgical procedure to correct the underlying cause of infection. Antibiotic regimens with little or no activity against Gram-negative rods or anaerobic Gram-negative rods are not considered acceptable (Holzheimer 2001).

Organisms found in acute infectious peritonitis include Escherichia coli, followed by the anaerobic Bacteroides fragilis group, Gram-positive anaerobic cocci, Enterococcus spp. and Klebsiella spp. (Shinagawa et al 1994). In postoperative peritonitis, the order of frequency of organisms was found to be Enterococcus spp. followed by Pseudomonas spp., Staphylococcus spp., E. coli, Enterobacter spp. and Klebsiella spp. (8%). In a study of 100 cases of infectious peritonitis in India, Shree et al (2013) found E. coli to be the predominant aerobic pathogen, followed by Klebsiella spp., while Bacteroides fragilis was the predominant anaerobe. Fungi were only recovered in 3 cases. Approximately two-thirds of E. coli and Klebsiella spp. were extended range beta-lactamase (ESBL) positive and a high level of resistance was observed for beta lactams, ciprofloxacin, amikacin, and ertapenem. In a study of 58 patients with infectious peritonitis in Mexico, Orozco et al (2006) found the following microorganisms in descending order of frequency: E. coli, Enterococcus spp., Streptococcus spp., Klebsiella spp., Pseudomonas spp., Enterobacter spp., Staphylococcus spp., Clostridium spp., Bacteroides spp. (only 1 patient) and Candida spp. (2 patients). In peritonitis associated with peritoneal dialysis, infectious organisms reflect skin and environmental contamination of the dialysis catheter and fluid, and Staphylococcus spp. (chiefly coagulase-negative) are the most common organisms found (Troidle et al 2006).

The antibiotics recommended to treat bacterial peritonitis vary with time and place, according to the local spectrum of infections and the prevalence of antibiotic-resistant organisms, as well as the development of succeeding generations of antibiotics intended to overcome resistance. Normally a broad-spectrum bactericidal antibiotic with activity against the majority of aerobic organisms is given intravenously, together with an antibiotic active against the principal anaerobic pathogens of the B. fragilis group. Although the intraperitoneal route of giving antibiotics may be the most effective for treating generalized infectious peritonitis of whatever cause, this has not usually been done except in the treatment of peritonitis associated with peritoneal dialysis, where there is already an intraperitoneal catheter in place.

### SUMMARY OF THE INVENTION

The purpose of the present invention is to provide the means for the optimal treatment of infectious peritonitis by both restoring defective peritoneal macrophage function by providing GM-CSF via the effective intraperitoneal route, and providing the most appropriate antibiotics to eliminate the causative organisms in this context, the antibiotics also being given by that route. The prime candidate antibiotic that is active against all the causative aerobic bacteria found in infectious peritonitis is fosfomycin. Fosfomycin is highly active against the aerobic genera Staphylococcus, Streptococcus, Neisseria, Escherichia, Proteus, Serratia, Salmonella, Shigella, Pseudomonas, Haemophilus and Vibrio, and active against Klebsiella and Enterobacter spp. (see for example, Rodriguez et al 1977). With respect to anaerobic genera, it is active against Peptostreptococcus (including Peptoniphilus, Finegoldia and Anaerococcus) and Fusobacterium, but not against bacteria of the B. fragilis group. Fosfomycin is highly effective against Enterobacteriaceae, including multidrug-resistant E. coli and ESLB-producing organisms.

Intraperitoneal administration of the antibiotic results in high local concentrations that are many times higher than the minimum inhibitory concentrations for even the organisms that are regarded as relatively resistant to fosfomycin. An advantage of fosfomycin is that it is not only remarkably non-toxic in terms of systemic adverse effects, but also that it lacks toxic effects on granulocytes, macrophages and dendritic cells, so that the effects of GM-CSF on these cells are unimpaired.

However, bacteria of the B. fragilis group are resistant to fosfomycin, so that in treating bacterial peritonitis in which bacteria of this group are reasonably expected to play a pathogenic role, one or more further antibiotics or antimicrobial agents with activity against this group must be provided to be given concomitantly by the same route. Non-limiting examples of such agents are metronidazole or carbapenems such as ertapenem, imipenem or meropenem.

In treating fungal peritonitis with intraperitoneal GM-CSF and one or more intraperitoneal antimicrobial agents, fosfomycin has no part to play and is substituted by antifungal agents, non-limiting examples of which are fluconazole or caspofungin.

The invention therefore consists of providing a means of optimizing the treatment of infectious peritonitis or intra-abdominal infection by providing GM-CSF administered intraperitoneally to improve peritoneal antimicrobial defense by restoring defective peritoneal macrophage function and recruiting further primed macrophages into the peritoneum without inducing systemic inflammation, while at the same time providing appropriate antimicrobial agents administered intraperitoneally to achieve high local bactericidal or fungicidal concentrations.

We have now surprisingly found that the preferred composition for use in accordance with the invention when used to treat secondary infectious peritonitis of bacterial origin, typically arising from a bowel perforation, is not only unexpectedly effective in treating the peritonitis but also reduces the occurrence of common complications that may arise during standard intravenous antibiotic treatment of this condition, such as local peritonitis or intra-abdominal abscess formation. Accordingly, the present invention provides the following pharmaceutical composition for intraperitoneal administration to treat secondary infectious peritonitis of bacterial origin with the avoidance of local peritonitis or intra-abdominal abscess formation:

In a first aspect, a composition is disclosed that comprises a granulocyte-macrophage colony-stimulating factor (GM-CSF) in combination with fosfomycin and any of the compounds selected from metronidazole, ertapenem, imipenem and meropenem for use in treatment, pre-emptive treatment or preventing secondary infectious peritonitis or intra-abdominal infection of bacterial origin in a subject, wherein the composition is for intraperitoneal administration.

In another aspect, a composition is disclosed that comprises granulocyte-macrophage colony-stimulating factor (GM-CSF) in combination with fosfomycin and any of the compounds selected from metronidazole, ertapenem, imipenem and meropenem for use in reducing the incidence of local peritonitis or intra-abdominal abscess, wherein the composition is for intraperitoneal administration.

In yet another aspect, a composition is disclosed that comprises granulocyte-macrophage colony-stimulating factor (GM-CSF) in combination with fosfomycin and any of the compounds selected from metronidazole ertapenem, imipenem and meropenem for use in postoperative treatment of perforated appendicitis, wherein the composition is for intraperitoneal administration.

In yet another aspect, a composition is disclosed that comprises granulocyte-macrophage colony-stimulating factor (GM-CSF) in combination with fosfomycin and metronidazole for use in the treatment, pre-emptive treatment or prevention of any indication selected from the list of secondary infectious peritonitis or intra-abdominal peritonitis or intra-abdominal infection of bacterial origin in a subject or reducing the incidence of local peritonitis in intra-abdominal abscess or postoperative treatment of appendicitis, wherein the composition is for intraperitoneal administration.

In yet another aspect, a composition is disclosed for use according to any one of the preceding aspects, wherein the composition comprises a dose of between 5 and 100 micrograms of GM-CSF, preferably 50 microgram of GM-CSF.

In yet another aspect, the composition is disclosed for use according to any one of the preceding aspects, wherein the composition comprises a dose of between 1 gram and 12 gram of fosfomycin, preferably 4 gram fosfomycin.

In yet another aspect, the composition is disclosed for use according to any one of the preceding aspects, wherein the composition comprises a dose of between 250 milligram and 3 gram of metronidazole, preferably 1 gram of metronidazole.

In yet another aspect, the composition is disclosed for use according to any one of the preceding aspects, wherein the composition is for administration in a volume of between 500 mL and 1500 mL.

In yet another aspect, the composition is disclosed for use according to any one of the preceding aspects, wherein the composition has a pH of between 6.5 and 8.0, preferably 7.4.

In yet another aspect, the composition is disclosed for use according to any of the preceding aspects, comprising a dose of 50 microgram of GM-CSF, 4 gram of fosfomycin and 1 gram of metronidazole in a total volume of aqueous solution of 500 mL for use in the treatment, pre-emptive treatment or prevention of secondary infectious peritonitis of bacterial origin, including the postoperative treatment of perforated appendicitis in a subject such that the incidence of local peritonitis or intra-abdominal abscess is reduced, wherein the composition is formulated for intraperitoneal administration.

In yet another aspect, the composition is disclosed for use according to any of the preceding aspects, wherein the composition is for administration between 1 and 6 times per day, such as 1 time, 2 times, 3 times, 4 times, 5 times or 6 times per day.

In yet another aspect, the composition is disclosed for use according to any of the preceding aspects, wherein the subject is a mammal.

In yet another aspect, the composition is disclosed for use according to the preceding aspect, wherein the subject is a human.

In yet another aspect, the composition is disclosed for use according to the preceding aspect, wherein the human is a child younger than 15 years of age.

In yet another aspect, the composition is disclosed for use according to some of the preceding aspects, wherein the human is an adult of 15 years of age or older.

In yet another aspect, a kit of parts is disclosed for use in the treatment, pre-emptive treatment or prophylaxis of infectious peritonitis or intra-abdominal infection of bacterial origin in a subject and reduce the incidence of local peritonitis or intra-abdominal abscess, and for use in the postoperative treatment of perforated appendicitis, where said kit comprises

(i) a first composition comprising GM-CSF;

(ii) a second composition comprising a pharmaceutically acceptable salt of fosfomycin; and

(iii) a third composition comprising a compound selected from metronidazole, ertapenem, imipenem or meropenem

wherein said first, second and third compositions are presented as solids or aqueous solutions formulated for intraperitoneal administration and are, optionally, mixed before such administration.

In yet another aspect, the kit of parts is disclosed for use according to the preceding aspect, wherein the third composition comprises metronidazole.

In yet another aspect, the kit of parts is disclosed for use according to the two preceding aspects, wherein the kit of part comprises 50 microgram of GM-CSF, 4 gram of fosfomycin and 1 gram of metronidazole in a total volume of aqueous solution of 500 mL.

In yet another aspect, a method is disclosed of treating, pre-emptively treating or preventing or ameliorating secondary infectious peritonitis or intra-abdominal infection of bacterial origin in a subject for reducing the incidence of local peritonitis or intra-abdominal abscess, including such as may occur in the postoperative treatment of perforated appendicitis, said method comprising administering into a peritoneal cavity a composition comprising granulocyte-macrophage colony-stimulating factor (GM-CSF) in combination with fosfomycin and any of the compounds selected from metronidazole, ertapenem, imipenem and meropenem.

In yet another aspect, the method is disclosed according to the preceding aspect, wherein the composition comprises metronidazole.

In yet another aspect, the method is disclosed according to the two preceding aspects, wherein the composition comprises between 5 and 100 micrograms of GM-CSF, between 1 gram and 12 gram of fosfomycin and between 250 milligram and 3 gram of metronidazole.

In yet another aspect, the method is disclosed according to the preceding aspect, wherein the composition comprises 50 microgram of GM-CSF, 4 gram of fosfomycin and 1 gram of metronidazole in a total volume of aqueous solution of 500 mL.

The compositions of the kit of parts are for simultaneous or concurrent intraperitoneal administration, but can, if circumstances so dictate, be administered separately or sequentially.

In the following detailed description of the invention, details of the scope of the invention and the meaning of certain terms used will be given, together with details of the practical performance of the invention.

### DETAILED DESCRIPTION OF THE INVENTION

The present invention relates to a composition for the treatment of infectious peritonitis or intra-abdominal infection. The essential features of the invention are 1) that the composition is for local administration into the peritoneal cavity by any appropriate means, but will typically be administered at operation, which will commonly be a laparoscopic intervention, or may also be administered via a catheter such as a Tenckhoff catheter implanted in the abdominal wall to terminate intraperitoneally; and 2) that the composition provides GM-CSF to improve peritoneal antimicrobial defense by restoring defective peritoneal macrophage function and recruiting further primed macrophages into the peritoneum without inducing systemic inflammation, while at the same time providing appropriate antimicrobial agents to achieve high local bactericidal or fungicidal concentrations. The combined effect of these features is to reduce the incidence of local peritonitis or intra-abdominal abscess.

The GM-CSF and the antibiotic agents of the composition may be formulated as a single composition for intraperitoneal administration. Preferably, however, the GM-CSF and the antibiotic agents are formulated in separate formulations for simultaneous, concurrent, separate or sequential administration into the peritoneal cavity. For simultaneous or concurrent administration, the GM-CSF and antibiotic agents can be mixed as aqueous solutions to form a single solution for instillation into the peritoneal cavity.

Accordingly, one of the aspects of the present invention provides a pharmaceutical composition comprising a recombinant, biologically active form of granulocyte-macrophage colony-stimulating factor (GM-CSF) together with fosfomycin and metronidazole for the treatment, pre-emptive treatment or prophylaxis of secondary infectious peritonitis or intra-abdominal infection of bacterial origin, wherein the composition is for intraperitoneal administration.

Another aspect of the present invention provides a kit of parts for use in the treatment, pre-emptive treatment or prophylaxis of infectious peritonitis or intra-abdominal infection of bacterial origin in a subject, where said kit comprises
(i) a first composition comprising a recombinant, biologically active form of granulocyte-macrophage colony-stimulating factor (GM-CSF);
(ii) a second composition comprising a suitable salt of fosfomycin disodium; and
(iii) a third composition comprising metronidazole

wherein said first, second and third compositions are presented as solids or solutions formulated for intraperitoneal administration.

The further embodiments described herein applies to both the aspect where the GM-CSF and the antimicrobial/antibiotic agent(s) are formulated as separate formulations and where the GM-CSF and the antimicrobial/antibiotic agent(s) are formulated as a single formulation.

The use of the invention is not intended to and does not substitute the necessary surgical procedures required to repair and control the underlying cause of the infection, such as the leakage of bacteria from a bowel perforation, but is fully compatible with such procedures and can be applied before, during and after such procedures.

The theoretical background for treatment is, as outlined above, that GM-CSF administered directly into the peritoneal cavity will restore the defective function of the peritoneal macrophages resulting from severe infection and will recruit further primed macrophages without giving rise to the general pro-inflammatory response of systemically administered GM-CSF. GM-CSF is a protein that is not expected to penetrate through the peritoneum to enter the blood stream, where its general systemic pro-inflammatory myelogenic effect would be potentially deleterious to the patient undergoing treatment for peritonitis.

Simultaneously, concurrently, separately or sequentially, one or more appropriate antibiotic agents are administered by the same, intraperitoneal route unless the antibiotic agents are co-formulated with the GM-CSF and administered as such. This enables considerably higher local concentrations of the agents to be achieved than those obtained after systemic administration. The preferred agent against the aerobic bacteria typically found in infectious peritonitis is fosfomycin, which active against all of these at the intraperitoneal concentrations obtained by intraperitoneal administration. It has the special advantage of being especially active against Enterobacteriaceae, including multidrug-resistant E. coli and ESBL-producing organisms, which are found in a high proportion of cases of infectious peritonitis (see e.g. Shree et al 2013). Fosfomycin is a small, highly diffusible molecule, showing high tissue penetration. Even is this case, intraperitoneal administration produces superior intraperitoneal levels to those obtained by intravenous administration (Tobudic et al 2012).

The combination of intraperitoneal application of GM-CSF with intraperitoneal application of appropriate antibiotic agents has a potent therapeutic effect in infectious peritonitis by providing the active ingredients of the compositions of the invention to the site where they are needed, thereby achieving a high local concentration and high local efficacy. At the same time, the reduced entry of these substances, especially GM-CSF, into the blood stream will reduce unwanted systemic effects. The therapeutic efficacy of the intraperitoneally applied compositions thus contrasts with the needlessly limited efficacy of the same active ingredients when given systemically.

The efficacy of intraperitoneal treatment here disclosed is emphasized by the fact that not only does it lead to a much more rapid resolution of secondary bacterial peritonitis than treatment with intravenous antibiotics according to widely accepted international protocols, but it also leads to a reduction in the frequency of subsequent local peritonitis or intra-abdominal abscess formation, which are complications that may occur during treatment with intravenous antibiotics. This is illustrated by Example 3 below.

Treatment with the compositions of the invention will be described in more detail below. Typically, the treatment of infectious peritonitis with the compositions of the invention will be as a single intraperitoneal dose given during the primary surgical or laparoscopic intervention, but in special circumstances the treatment may be prolonged and further intraperitoneal doses may be given via an indwelling catheter. Conventional antimicrobial treatment of infectious peritonitis has customarily been continued until the patient's fever has subsided and the white blood cell count returned to normal. If the patient's condition does not improve on the initial intraperitoneal dosage regimen, the dosage can be increased by, for example, doubling the dose, in consideration of the lack of serious adverse effects.

### Active Ingredients of the Compositions of the Invention

Compositions according to the present invention comprise essentially a recombinant, biologically active form of granulocyte-macrophage-colony stimulating factor (GM-CSF) and the preferred antibiotic agents fosfomycin and metronidazole. Metronidazole may be substituted by ertapenem or imipenem or meropenem.

### Granulocyte-Macrophage Colony-Stimulating Factor (GM-CSF)

GM-CSF is a member of the family of colony-stimulating factors (CSFs), which are glycoproteins that stimulate the proliferation and maturation of hematopoietic progenitors and enhance the functional activity of mature effector cells. In brief, at the level of the immature cells, CSFs ensure the self-renewal of the staminal pool and activate the first stage of hematopoietic differentiation. In the subsequent stage, when cell proliferation is associated with a progressive acquisition of the characteristics of the mature cells, they enormously enhance the number of differentiating cells. In the terminal stage, they stimulate the circulation and the activation of mature cells.

Mature GM-CSF is a monomeric protein of 127 amino-acid residues with several potential glycosylation sites. The variable degree of glycosylation results in a molecular weight range between 14 kDa and 35 kDa. Non-glycosylated and glycosylated GM-CSF show similar activity in vitro (Cebon et al 1990). The crystallographic analysis of GM-CSF revealed a barrel-shaped structure composed of four short alpha helices (Diederichs et al 1991). There are two known sequence variants of GM-CSF. The active form of the GM-CSF protein is found extracellularly as a homodimer in vivo.

GM-CSF exerts its biological activity by binding to its receptor. The most important sites of GM-CSF receptor (GM-CSF-R) expression are on the cell surface of myeloid cells, such as macrophages types I and II, epithelial cells and endothelial cells, whereas lymphocytes are GM-CSF-R negative. The native receptor is composed of alpha and beta subunits. The alpha subunit imparts ligand specificity and binds GM-CSF with nanomolar affinity. The beta subunit is also part of the interleukin-3 and interleukin-5 receptor complexes and, in association with the GM-CSF-R alpha subunit and GM-CSF, leads to the formation of a complex with picomolar binding affinity (Hayashida et al 1990). The binding domains on GM-CSF for the receptor have been mapped: GM-CSF interacts with the beta subunit of its receptor via a very restricted region in the first alpha helix of GM-CSF (Shanafelt et al 1991a; b; Lopez et al 1991). Binding to the alpha subunit could be mapped to the third alpha helix, helix C, the initial residues of the loop joining helices C and D, and to the carboxyterminal tail of GM-CSF (Brown et al 1994).

Formation of the GM-CSF trimeric receptor complex leads to the activation of complex signaling cascades involving molecules of the JAK/STAT families, She, Ras, Raf, the MAP kinases, phosphatidylinositol-3-kinase and NFkB, finally leading to the transcription of c-myc, c-fos and c-jun. Activation is mainly induced by the beta subunit of the receptor (Hayashida et al 1990; Kitamura et al 1991; Sato et al 1993). The shared beta subunit is also responsible for the overlapping functions exerted by IL-3, IL-5 and GM-CSF (reviewed by de Groot et al 1998).

In addition to its stimulating activity on hemopoietic growth and differentiation, GM-CSF acts as a pro-inflammatory cytokine. Macrophages, e.g. macrophages types I & II and monocytes, as well as neutrophils and eosinophils, are activated by GM-CSF, resulting in the release of other cytokines and chemokines and matrix-degrading proteases, as well as increased expression of HLA and cell adhesion molecules or receptors for CC-chemokines. This in turn leads to increased chemotaxis of inflammatory cells into inflamed tissue.

For practical purposes, the GM-CSF preparations to be used in the present invention will not be purified native human GM-CSF, which could of course be used if it were available in sufficient quantity and problems of possible viral contamination were overcome, but human GM-CSF prepared in vitro by recombinant DNA technology. The preparation of recombinant human GM-CSF (rhGM-CSF) in mammalian cells has been described (Wong et al 1985; Kaushansky et al 1986). Similar work has led to the production of rhGM-CSF with the non-proprietary name regramostim in Chinese hamster ovarian (CHO) cells (first reported by Moonen et al 1987). The expression of rhGM-CSF in Saccharomyces cerevisiae was reported by Cantrell et al (1985), leading to the preparation known by the non-proprietary name sargramostim. Sargramostim differs from endogenous human GM-CSF in having a leucine residue instead of an arginine residue at position 23 of the mature protein and is less glycosylated than either endogenous human GM-CSF or regramostim (Armitage 1998). The expression of rhGM-CSF in Escherichia coli was reported by Burgess et al (1987), leading to the preparation known by the non-proprietary name molgramostim, which is not glycosylated. All three rhGM-CSF preparations, regramostim, sargramostim and molgramostim can be used in the present invention, but only the last two are currently available. The said rhGM-CSF preparations all show the biological activities mentioned above. When the term “recombinant, biologically active GM-CSF” is used herein, it means, in the context of its use in human subjects, the preparations regramostim, sargramostim or molgramostim, of which the preferred form is molgramostim.

The composition for use as disclosed herein comprises GM-CSF in its biologically active form. Thus, pre-GM-CSF does not fall under the term GM-CSF. A person skilled in the art based on a common general knowledge can determine what is a biologically active from of GM-CSF.

The amino-acid sequence of the principal form of human GM-CSF as a mature protein (Example 1, SEQ ID NO: 1) is:

APARSPSPST QPWEHVNAIQ EARRLLNLSR DTAAEMNETV
EVISEMFDLQ EPTCLQTRLE LYKQGLRGSL TKLKGPLTMM
ASHYKQHCPP TPETSCATQI ITFESFKENL KDFLLVIPFD
CWEPVQE.

Compositions for use according to the present invention may comprise recombinant, biologically active GM-CSF at doses of 5 microgram to 100 microgram, such that when added to 500 mL to 1500 mL of fluid suitable for instillation into the peritoneal cavity, concentrations of 3.33 microgram per liter to 200 microgram per liter can be obtained.

### Fosfomycin

Fosfomycin is the international non-proprietary name of a broad-spectrum antibiotic isolated and characterized in 1969 from Streptomyces fradiae strains under the name phosphomycin or phosphonomycin (Hendlin et al 1969). Its structure was determined to be (−)(IR, 2S)-1,2-epoxypropylphosphonic acid (Christensen et al 1969), with the systematic (IUPAC) name [(2R,3S)-3-methyloxiran-2-yl]phosphonic acid and a formula weight of 138.1 Da. Fosfomycin is bactericidal and inhibits bacterial cell wall biosynthesis by inactivating the enzyme UDP-N-acetylglucosamine-3-enolpyruvyltransferase, also known as MurA (Brown et al 1995). This enzyme catalyzes the committed step in peptidoglycan biosynthesis, the ligation of phosphoenolpyruvate to the 3′-hydroxyl group of UDP-N-acetylglucosamine to form N-acetylmuramic acid. Fosfomycin is a phosphoenolpyruvate analogue that inhibits MurA by alkylating an active site cysteine residue. The antibiotic enters the bacterial cell via the glycerophosphate transporter.

Given this mechanism of action, fosfomycin has a broad bactericidal spectrum, being active against aerobic genera such as Staphylococcus, Streptococcus, Neisseria, Escherichia, Proteus (indole-negative), Serratia, Salmonella, Shigella, Pseudomonas, Haemophilus, and Vibrio, less active against indole-positive Proteus spp., Klebsiella and Enterobacter spp. It is known to be active against the anaerobic genera Peptostreptococcus (including Peptoniphilus, Finegoldia and Anaerococcus) and Fusobacterium.

There is a low prevalence of bacterial resistance to fosfomycin in the community, and studies of the prevalence of resistant bacteria after the introduction of fosfomycin have shown either no increase or only a modest increase in the prevalence of resistant organisms. However, prolonged exposure to the antibiotic may enable bacteria to evolve resistance by selection of mutants that lack the glycerophosphate transporter pathway. Alternative mechanisms of resistance involve the loss of the inducible hexose phosphate transporter, a Cys-Asp mutation in MurAS, or acquistion of plasmids coding for the fosfomycin inactivating enzymes fosA and fosB (in addition to the chromosomal fosX in Listeria monocytogenes). The mutant strains may, however, also show reduced pathogenicity (Karageorgopoulos et al 2012). This may explain why the emergence of bacterial resistance is seen on prolonged exposure in vitro, but much less frequently in vivo. The appearance of resistant bacterial strains in controlled clinical trials of orally or intravenously administered fosfomycin has been 3.0% overall, with a maximum of 15% for Pseudomonas aeruginosa. In general, fosfomycin is seen to be a valuable addition to the therapeutic armament against multidrug-resistant organisms.

Fosfomycin has proved to be remarkably non-toxic to mammalian cells and organs, despite fosfomycin disodium being used at intravenous doses of up to 0.5 g/kg/day in human patients. Here the limiting factor is overload with the counter-ion rather than any toxic effect of the antibiotic. Indeed, fosfomycin has been found to exert a protective effect against the toxic action of other antibiotics, immunosuppressive or chemotherapeutic agents such as aminoglycosides, vancomycin, amphotericin B, polymyxin, cyclosporin and cisplatin (Gobernado 2003). As additional effects, it has the capacity to favor phagocytosis and act as an immunomodulator. It is accumulated by polymorphonuclear leukocytes to reach concentrations that are twice those of the extracellular fluid, but does not affect their cellular functions, while exerting a bactericidal effect on Staphylococcus aureus. The chief adverse effects are gastric irritation from orally administered fosfomycin disodium, evidence of allergy in the form of transient rashes (0.3% of cases) and eosinophilia (0.2%), and transiently raised liver enzymes (0.3% of cases) (Gobernado 2003).

Fosfomycin shows a considerable synergism in bactericidal effect on a large number of strains of organisms from the susceptible genera mentioned, when used in combination with a large number of antibiotics of the penicillin, cephalosporin, aminoglycoside, macrolide and lincosamide types. While early studies showed a synergistic effect on about 70-100% of tested strains for various antibiotic combinations, subsequent more extensive studies showed synergy rates of 36-74%. The remaining strains showed merely additive effects and an inhibitory effect was only seen in one or two individual antibiotic combinations on an individual bacterial strain (Gobernado 2003). However, in a specific study of an animal model of infectious peritonitis, fosfomycin showed synergy and high in-vivo efficacy with imipenem (Pachon-Ibanez et al 2011). The fact that fosfomycin shows synergy with many individual antibiotics and indeed abrogates the toxicity of many other antibiotics, including the nephrotoxicity and ototoxicity of the aminoglycosides, favors the use of fosfomycin in combination with other antibiotics to produce a potent bactericidal action and compensate for any development of fosfomycin resistance during more prolonged treatment.

The principal forms of fosfomycin that come within the scope of this invention are:
i) Fosfomycin disodium, formula weight 182.0 Da, pH of 5% solution 9.0-10.5. This salt is highly soluble in water, but is locally irritant if un-neutralized.
ii) Fosfomycin trometamol, formula weight 259.2 Da, pH of 5% solution 3.5-5.5. This salt is highly soluble in water and is well tolerated when given orally.

When the name “fosfomycin” is used herein, it refers to an inorganic or organic salt of fosfomycin as exemplified by the principal forms above, and the dose of fosfomycin refers to the amount of the free acid form of fosfomycin present in the salt.

Compositions for use according to the present invention may comprise fosfomycin such that single doses of 1 gram to 12 gram are given. These can be dissolved in a total volume of 500 mL to 1500 mL of fluid suitable for instillation into the peritoneal cavity. A preferred concentration of instilled fosfomycin is 8 gram per liter, which can provide for isotonicity of the solution. In no case will the daily dose administered intraperitoneally exceed the maximum recommended daily intravenous dose, in the range of 24 gram to 32 gram.

### Metronidazole

This semi-synthetic antibiotic is active against a number of anaerobic bacteria including bacteria of the B. fragilis group and Fusobacterium spp. It has been used systemically in combination with systemic fosfomycin for prophylaxis against wound infection after abdominal surgery. Its intraperitoneal use in the treatment of secondary bacterial peritonitis in accordance with the present invention has not previously been studied. Compositions for intraperitoneal administration according to the present invention may comprise metronidazole such that single doses are typically about one quarter of the dose of fosfomycin and hence in the theoretical range of 250 milligram to 3 gram of metronidazole, but in no case will the maximum recommended daily intravenous dose of metronidazole of 2 gram be exceeded.

### Carbapenems

The carbapenems are members of the beta-lactam class of antibiotics, which, like the penicillins and cephalosporins, exert a bactericidal action by inhibiting bacterial cell wall synthesis by a different mechanism than that of fosfomycin. Hence the beta-lactam antibiotics, including the carbapenems, characteristically show a synergic bactericidal action with fosfomycin on most bacterial species against which both types of antibiotic are active. However, the carbapenems exhibit a broader spectrum of activity than most penicillins and cephalosporins, being active against anaerobic bacteria such as the B. fragilis group, Prevotella spp. and Fusobacterium spp. They can hence be used in place of metronidazole to exert a bactericidal action on the anaerobic bacteria that are largely insensitive to fosfomycin. Compositions according to the present invention may comprise ertapenem, or meropenem, or imipenem, such that single doses are in the same range as that for metronidazole.

### Indications for Use

The indications for the use of compositions according to the present invention are suspected or confirmed, incipient or established infectious peritonitis or intra-abdominal infection, or a high imminent risk of the same, as determined by the attending surgeon or physician according to the criteria known to the skilled person. Typically, the indication for use will be established or confirmed by laparoscopy or other surgical intervention, and the initial dose of the composition, which may be the only dose that is necessary, will be given during the surgical procedure.

Preferably, an indication in accordance with the present invention is a secondary infectious peritonitis.

Preferably, an indication is local infectious peritonitis.

Preferably, an indication according to the present invention is perforated appendicitis. A perforated appendix was defined as an appendectomy during which the operating surgeon or the supervisor determined the need for an intravenous postoperative course of antimicrobial agents. This usually included a visible appendix perforation, free intra-abdominal pus, visible feces, and/or an abscess.

In some embodiments the composition is used for local treatment of postoperative bacterial infections in the peritoneal cavity.

The effect of the use of the compositions of the invention is assessed by the attending clinician in accordance with the evolution of the affected individual's clinical signs and symptoms, usually taking into account such biomarkers as body temperature and white blood-cell count.

In one embodiment of the present invention, the subject in need of being administered with a composition of the invention is a mammal in need of treatment, pre-emptive treatment or prevention of infectious peritonitis or intra-abdominal infection. The recombinant, biologically active form of the GM-CSF to be used in a particular mammal is a form of GM-CSF that is biologically active in that mammalian species, which, when the species is not a human or non-human primate species, will mean that the GM-CSF will be that of the same or closely related mammalian species.

In one embodiment, the compositions of the present invention are for use in a patient who is not a peritoneal dialysis patient, or wherein the peritonitis is not caused by the presence of a peritoneal catheter.

In one embodiment, the mammal is a human subject. In one embodiment, the human subject is a child younger than 15 years of age. In one embodiment, the human subject is an adult of 15 years of age or older.

### Formulations

Pharmaceutical compositions or formulations for use in the present invention comprise recombinant, biologically active GM-CSF together with one or more antibiotic agents for the treatment, pre-emptive treatment or prophylaxis of infectious peritonitis or intra-abdominal infection, wherein the composition is for intraperitoneal administration. In a preferred embodiment, the composition comprising GM-CSF further comprises fosfomycin and metronidazole as the antibiotic agents. In further embodiments, the metronidazole of the preferred embodiment is replaced by ertapenem, or meropenem, or imipenem. Such compositions or formulations may be dissolved in a pharmaceutically acceptable carrier, preferably an aqueous carrier or diluent. A variety of aqueous carriers may be used, including, but not limited to water for injection, 0.9% saline, buffered saline, physiologically compatible buffers and the like. The compositions may be sterilized by conventional techniques well known to those skilled in the art, with the proviso that the heat-labile GM-CSF is not to be subjected to heat sterilization but is sterilized by sterile filtration. The resulting aqueous solutions may be packaged for use or filtered under aseptic conditions and freeze-dried, the freeze-dried preparation being dissolved in sterile water for injection or other sterile aqueous solution prior to administration. In one embodiment, a freeze-dried preparation comprising recombinant, biologically active GM-CSF such as molgramostim may be pre-packaged, for example in a single dose unit. In another embodiment, the constituents of the composition are supplied in a kit containing the individual constituents GM-CSF and fosfomycin in dry form in separate vials, together with a sterile metronidazole solution in a third container. This makes it possible for the dose of each that is to be administered intraperitoneally to be varied, if the clinician so desires.

The compositions may contain pharmaceutically acceptable auxiliary substances or adjuvants, including, without limitation, pH-adjusting and buffering agents and/or tonicity adjusting agents, such as, for example, succinic acid, citric acid, sodium acetate, sodium bicarbonate, sodium lactate, sodium chloride, potassium chloride, calcium chloride, or, for GM-CSF, stabilizing agents and agents permitting ready redissolution of freeze-dried proteins, such as mannitol or other sugars, polyethylene glycol and virus-free or recombinantly produced human serum albumin.

The individual components of the compositions according to the present invention are formulated such that, when solutions of them are mixed for intraperitoneal administration, the pH value of the resulting combined solution is as close as possible to the physiological pH value of 7.4, and will not lie outside a range of 6.5 to 8.0.

Preferably, the composition according to the invention comprises 50 microgram freeze-dried molgramostim. Preferably, the composition comprises 4 gram of fosfomycin as dry fosfomycin disodium powder. Preferably, the composition comprises 1 gram of metronidazole injection USP. The pH of the solution is 7.46-7.62. The amount of the composition is calculated per an adult with body weight of 50 kg to 110 kg. The composition may be easily scaled up or down to fit, for example a child or an adult with the body weight higher than 110 kg. In such case, the amount of freeze-dried molgramostim is between 0.45 microgram and 1 microgram per kilogram of body weight; the amount of fosfomycin dry powder is between 0.03 gram and 0.08 gram per kilogram of body weight, and the amount of metronidazole is between 0.009 and 0.02 gram per kilogram of body weight.

Example 2 below gives a specimen formulation of the preferred embodiment of the present invention suitable for treating bacterial peritonitis.

### Administration

The compositions of the present invention are for intraperitoneal use and may be given by methods conventionally used in the art for instillation into the peritoneal cavity. This will typically be done at surgery, whether it be open or laparoscopic abdominal surgery. It may also be performed by techniques similar to those used for continuous or chronic peritoneal dialysis (CPD), especially in cases where treatment is expected to be prolonged. There are two types of CPD therapy: continuous ambulatory peritoneal dialysis (CAPD) and continuous cycling peritoneal dialysis (CCPD). The former modality involves manual dialysis exchanges performed throughout the day (usually three to five) while the latter modality is performed continuously for approximately 8 to 10 hours at night using an automated cycling device. The method giving most flexibility will be similar to CAPD, with the difference that the patient is typically recumbent, not ambulatory, and the manual fluid exchanges are spread throughout the 24 hours of the day. Typically fluid volumes of 1500 mL to 3000 mL are used in fluid exchanges in adult patients. However, for the treatment of infectious peritonitis the purpose is not to dialyze, but to provide sufficient fluid containing the compositions of the invention to allow them to reach all parts of the peritoneal cavity. Hence smaller volumes can be used, such as 500 mL, 1000 mL, and 1500 mL.

Administration of solutions containing the compositions of the invention may, for example, comprise the steps of:

a. Inserting a catheter, such as a Tenckhoff catheter or similar device, through the abdominal wall to terminate with its outlet positioned in the peritoneal cavity at an appropriate site determined by the treating surgeon. This can conveniently be done at the time of surgical exploration to determine and treat the underlying cause of the infectious peritonitis, such as a bowel perforation, but can also be done before or after that by means of laparoscopy or even by a percutaneous technique.

b. Instilling a suitable volume of suitable fluid containing the composition to be used at the concentration determined by the attending clinician.

c. The dwell time of the fluid in the peritoneal cavity may be from 4 hours to 24 hours, corresponding to 6 exchanges or one exchange of fluid per day.

A preferred total volume for administering a single dose of the composition is about 500 mL or a fraction of a milliliter thereover, as this has been shown to be well tolerated and effective in human adult subjects of 50 kg to 110 kg body weight. For administration to children, the volume is scaled down in proportion to body weight, maintaining the concentration of the active components constant, so that the dose of these is also scaled to body weight. Similarly, the volume and dose administered can be scaled up in proportion to body weight for individuals weighing more than 110 kg. Preferably, the total volume for administering single dose of the composition is between about 4.5 mL/kg of body weight to 10 mL/kg of body weight.

Preferably, the administration of the composition of the invention is followed by orally administered antibiotic agents. Preferably, the antibiotic agent is a single antibiotic of a broad spectrum or a combination of at least two antibiotic agents. Preferably, at least two antibiotic agents are amoxicillin, clavulanic acid and metronidazole.

Preferably, a dosage of the antibiotic agents administered orally is: 500 mg amoxicillin combined with 125 mg clavulanic acid and 500 mg metronidazole. This is a dosage calculated for an adult of body weight between 50 kg to 110 kg. It shall be understood that the dosage may be adjusted accordingly if a patient has a body weight higher than 110 kg or lower than 50 kg.

The orally administered antibiotics are given for at least three consecutive days. The oral antibiotic agents are given at least once a day, preferably three times a day. In Example 3 below, the oral antimicrobial agents were administered three times daily for three consecutive days.

### Dosage

By “effective amount” of the compositions of the present invention is meant a dose, which, when administered intraperitoneally to a subject in need thereof, achieves a concentration in the intraperitoneal solution which has a beneficial biological effect in the treatment, pre-emptive treatment or prophylaxis of infectious peritonitis or intra-abdominal infection.

Compositions according to the present invention comprise recombinant, biologically active GM-CSF in an effective amount, which may be from 5 microgram to 100 microgram per dose, such that when a dose is added to 500 mL to 1500 mL of fluid suitable for instillation into the peritoneal cavity, concentrations of 3.33 microgram per liter to 200 microgram per liter can be obtained. The composition for use in the present invention comprise preferably from 10 microgram to 100 microgram per dose, such as 30 to 70 microgram per dose, such as 40 to 60 microgram per dose, such as 5 to 10 microgram per dose.

The composition for use according to the invention, comprises a dose of from 1 gram to 12 gram of fosfomycin, such as 2 to 10 gram of fosfomycin, such as 3 to 9 gram of fosfomycin, such as 4 to 8 gram of fosfomycin, such as 5 to 6 gram of fosfomycin, preferably 4 gram of fosfomycin.

The composition for use according to the invention, comprises a dose of between 250 milligram and 3 gram of metronidazole, such as from 500 milligram to 2 gram of metronidazole, preferably 1 gram of metronidazole.

If more than an initial single dose is needed, subsequent doses can be administered once a day, twice a day, three times a day, four times a day, five times a day or six times a day, through an indwelling catheter.

Duration of dosage will typically range from a single initial dose given at surgery, to a duration of treatment that may extend for as much as up to 14 days, or for as long as the attending clinician deems necessary.

The compositions according to the present invention comprise fosfomycin such that single doses of 1 gram to 12 gram are given. These can be dissolved in a total volume of 500 mL to 1500 mL of fluid suitable for instillation into the peritoneal cavity. A preferred concentration of instilled fosfomycin is 8 gram per liter, which can provide for isotonicity of the solution. In no case will the daily dose administered intraperitoneally exceed 32 gram.

The compositions according to the present invention may comprise metronidazole such that single doses of metronidazole are typically about one quarter of the dose of fosfomycin and hence in the theoretical range of 250 milligram to 3 gram of metronidazole, but in no case will the maximum recommended daily intravenous dose of metronidazole of 2 gram be exceeded.

The compositions according to the present invention that comprise ertapenem, or meropenem, or imipenem in substitution for metronidazole, comprise an amount of the substituting antibiotic such that single dose of said antibiotic is in the same range as that for metronidazole.

### EXAMPLES

The following non-limiting examples further illustrate the preferred composition and practice of present invention.

### Example 1

mature human GM-CSF
>sp|P04141|18-144
SEQ ID NO: 1
APARSPSPSTQPWEHVNAIQEARRLLNLSRDTAAEMNETVEVISEMFDLQ
EPTCLQTRLELYKQGLRGSLTKLKGPLTMMASHYKQHCPPTPETSCATQI
ITFESFKENLKDFLLVIPFDCWEPVQE

### Example 2

Composition of a preferred composition that provides an effective dose of recombinant, biologically active GM-CSF (molgramostim) together with fosfomycin and metronidazole when given intraperitoneally to treat secondary bacterial peritonitis in a human subject:

The composition is presented as three components. The first component consists of 50 microgram of freeze-dried molgramostim, with mannitol, PEG-4000, recombinant human albumin, disodium hydrogen phosphate and citric acid as excipients in a capped, evacuated glass vial. The second component consists of 4 gram of fosfomycin as dry fosfomycin disodium powder with succinic acid as an excipient in a capped, evacuated glass vial. The third component consists of 1 gram of metronidazole injection USP, with sodium chloride, disodium hydrogen phosphate and citric acid as excipients dissolved in sterile water for injection. The component is contained in two plastic infusion bags, each containing 500 milligram of metronidazole in 100 mL of solution. The molgramostim is dissolved on 0.2 mL of sterile water for injection; the fosfomycin is dissolved in 300 ml of sterile water for injection; the fosfomycin and metronidazole solutions are mixed and the molgramostim solution washed in immediately prior to instillation of the resulting volume of 500.2 mL of solution into the peritoneal cavity. The pH of the solution is 7.46-7.62.

### Example 3

### Clinical Testing of the Preferred Composition of the Invention Described in Example 2

A quasi-randomized prospective clinical trial was performed to test the efficacy of the preferred composition of the invention. Appropriate permissions to conduct the trial were obtained from the Danish Medicines Agency and the Scientific Ethics Committee, and the trial was supervised by the local Good Clinical Practice Unit. The intervention group consisted of 6 participants who completed the trial. They were given the trial composition described in Example 2 intraperitoneally, the composition being left in the abdominal cavity, immediately after laparoscopic appendectomy for appendicitis with a perforated appendix. Postoperatively, they received antimicrobial agents orally. The control group also consisted of 6 participants who completed the trial and had a perforated appendix removed at laparoscopy. These received intravenous antimicrobial agents both during surgery and postoperatively according to the approved local treatment protocol (intravenous metronidazole and either intravenous cefuroxime or intravenous piperacillin/tazobactam). The total length of stay within 30 days after the operation was evaluated for both groups, the time of discharge from hospital being determined by uniform objective criteria.

The control group received standard intravenous antibiotic agents during surgery (1 g metronidazole and either 1 g cefuroxime or 4 g piperacillin/500 mg tazobactam). Postoperatively, participants received three days of intravenously administered antibiotic agents: 4 g piperacillin/500 mg tazobactam and 500 g metronidazole. These doses were administered three times daily for a minimum of three days.

The detailed data for each patient and the results are summarized in Table 1 below.

TABLE 1

| | | Intervention | | Control | | |
|---|---|---|---|---|---|---|
| Admission | | group (n = 7) | | group (n = 6) | | p-value |
| Age, years | | 52 | (21-73) | 28 | (18-55) | 0.13† |
| Sex, female | | 4 | (57%) | 1 | (17%) | 0.27‡ |
| Height, cm | | 173 | (163-185) | 185 | (168-187) | 0.06† |
| Weight, kg | | 82 | (65-105) | 92 | (73-107) | 0.78† |
| Body Mass | | 29 | (24-34) | 28 | (21-33) | 0.63† |
| Index, kg/m² | | | | | | |
| ASA | I: | 4 | (57%) | 6 | (100%) | 0.19‡ |
| score | II: | 3 | (43%) | 0 | (0%) | |

Preoperative antimicrobial agents*

| | | | | | |
|---|---|---|---|---|---|
| Times | 2 | (0-8) | 2 | (0-4) | 0.54† |
| administered | | | | | |

Surgery

| | | | | | |
|---|---|---|---|---|---|
| Length, | 01:22 | (01:03-02:33) | 01:20 | (00:38-02:37) | 0.78† |
| hours:minutes | | | | | |

Table 1 demonstrates demographics of the 13 included patients for continuous variables in median (range) and for categorical variables in numbers (percent). One patient withdrew consent on postoperative day 3.
ASA: American Society of Anesthesiologists [14].
*Included the following antimicrobial agents: ampicillin, cefuroxime, gentamicin, piperacillin/tazobactam, and metronidazole.
†Mann-Whitney U-test,
‡Fisher's exact test.

Harms and adverse events, the gastrointestinal quality of life index, postoperative complications, and convalescence were evaluated over the same period and are summarized in Table 2.

TABLE 2
Table 2: The median (range) scores of the Gastrointestinal Quality of Life Index (GIQLI) are
given overall and for each item of the intervention and the control group postoperatively.
The maximal total scores are 144 for GIQLI, 76 for symptoms, 28 for physical function, 12
for emotions, 14 for social function, and the minimal score for medical treatment is 1.

| | 10^{th} postoperative day | | | | 30^{th} postoperative day | | | |
|---|---|---|---|---|---|---|---|---|
| | Intervention | | Control | | Intervention | | Control | |
| GIQLI | group (n = 6) | | group (n = 6) | | group (n = 6) | | group (n = 4) | |
| Total | 112 | (102-119) | 102 | (86-115) | 118 | (73-126) | 120 | (114-120) |
| Symptoms | 62 | (57-69) | 55 | (45-66) | 68 | (43-72) | 69 | (68-69) |
| Emotions | 12 | (8-15) | 11 | (5-14) | 13 | (8-14) | 14 | (12-15) |
| Physical function | 18 | (14-26) | 20 | (15-23) | 21 | (9-28) | 23 | (21-27) |
| Social function | 14 | (11-16) | 13 | (10-14) | 9.5 | (6-12) | 9 | (8-12) |
| Medical treatment | 4 | (3-4) | 3 | (2-4) | 3.5 | (1-4) | 4 | (3-4) |

### Statistical Methods

Data were analyzed by means of the SAS Enterprise Guide 7.1 (SAS Institute Inc., USA). In short, continuous numerical values are reported as median and range if not normally distributed. Non-normally distributed continuous data are analyzed with non-parametric statistics (Mann-Whitney U-test). Binary, categorical data are reported as numbers and proportions in %. These were analyzed with the chi-squared-test, and if any of the expected cell counts were <5, then p-values from Fisher's exact test are reported. A p-value≤1.05 was considered statistically significant.

The results showed that the total postoperative length of stay of the intervention group was markedly shortened to a median of 13 hours (range 2-21 hours) versus a median length of stay of 84 hours (range 67-169 hours, p=0.017) for the control group. Comparable harms, gastrointestinal quality of life index scores, and duration of convalescence were found in the two groups. Whereas there were no serious adverse events or complications in the intervention group, there were two separate cases of intra-abdominal abscess in the control group.

It is the opinion of the inventors that this clinical trial demonstrates the unexpected efficacy of the preferred composition according to the present invention in the treatment of secondary bacterial peritonitis following appendicitis with perforation of the appendix. The intraperitoneal treatment with the composition of the invention was so effective that a single dose given at operation shortened postoperative hospitalization to an average of about one sixth of that resulting from treatment with a current nationally and internationally approved intravenous antibiotic regimen.

Remarkably, there were no cases of local peritonitis or intra-abdominal abscess in the treatment group, whereas two such cases occurred in the control group. In the inventors' opinion, this demonstrates that the intraperitoneal treatment of the invention is efficacious in preventing this complication that too often occurs during conventional postoperative therapy for peritonitis with intravenous antibiotics. The intraperitoneal treatment not only improves the efficacy of the local immune response in secondary peritonitis by providing an appropriate stimulus from the locally administered GM-CSF (without stimulating an untoward systemic inflammatory response), but also ensures an appropriate local distribution of effective antibiotics at high local concentration, which reaches all parts of the peritoneal cavity and prevents islands of local peritonitis or intra-abdominal abscess from being left to develop in circumstances in which systemically administered antibiotics do not reach a sufficient local concentration to be effective in all regions of the peritoneal cavity.

### REFERENCES

Armitage J O (1998) Emerging applications of recombinant human granulocyte-macrophage colony-stimulating factor. Blood 92:4491-4508.
Austin O M, Redmond H P, Watson W G, Cunney R J, Grace P A, Bouchier-Hayes D
(1995) The beneficial effects of immunostimulation in posttraumatic sepsis. J Surg Res 59:446-449.
Barsig J, Bundschuh D S, Hartung T, Bauhofer A, Sauer A, Wendel A (1996) Control of fecal peritoneal infection in mice by colony-stimulating factors. J Infect Dis 174:790-799.
Brown C B, Pihl C E, Kaushansky K (1994) Mapping of human granulocyte-macrophage-colony-stimulating-factor domains interacting with the human granulocyte-macrophage-colony-stimulating-factor-receptor alpha-subunit. Eur J Biochem 225:873-880.
Brown E D, Vivas E I, Walsh C T, Kolter R (1995) MurA (MurZ), the enzyme that catalyzes the first committed step in peptidoglycan biosynthesis, is essential in Escherichia coli. J Bacteriol 177:4194-4197.
Burgess A W, Begley C G, Johnson G R, Lopez A F, Williamson D J, Mermod J J, Simpson R J, Schmitz A, DeLamarter J F (1987) Purification and properties of bacterially synthesized human granulocyte-macrophage colony stimulating factor. Blood 69:43-51.
Cantrell M A, Anderson D, Cerretti D P, Price V, McKereghan K, Tushinski R J, Mochizuki D Y, Larsen A, Grabstein K, Gillis S, et al (1985) Cloning, sequence, and expression of a human granulocyte/macrophage colony-stimulating factor. Proc Natl Acad Sci USA 82:6250-6254.
Cebon J, Nicola N, Ward M, Gardner I, Dempsey P, Layton J, Duhrsen U, Burgess A W, Nice E, Morstyn G (1990) Granulocyte-macrophage colony stimulating factor from human lymphocytes. The effect of glycosylation on receptor binding and biological activity. J Biol Chem 265:4483-4491.
Christensen B G, Leanza W J, Beattie T R, Patchett A A, Arison B H, Ormond R E, Kuehl F A Jr, Albers-Schonberg G, Jardetzky O (1969) Phosphonomycin: structure and synthesis. Science 166:123-125.
Daley B J (2013) Peritonitis and abdominal sepsis. emedicine.medscape.com/article/180234-overview.
Demetri G D, Zenzie B W, Rheinwald J G, Griffin J D (1989) Expression of colony-stimulating factor genes by normal human mesothelial cells and human malignant mesothelioma cells lines in vitro. Blood 74:940-946.
Diederichs K, Jacques S, Boone T, Karplus P A (1991) Low-resolution structure of recombinant human granulocyte-macrophage colony stimulating factor. J Mol Biol 221:55-60.
Gennari R, Alexander J W, Gianotti L, Eaves-Pyles T, Hartmann S (1994) Granulocyte macrophage colony-stimulating factor improves survival in two models of gut-derived sepsis by improving gut barrier function and modulating bacterial clearance. Ann Surg 220:68-76.
Gobernado M (2003) Fosfomycin. Rev Esp Quimioter 16:15-40.
de Groot R P, Coffer P J, Koenderman L (1998) Regulation of proliferation, differentiation and survival by the IL-3/IL-5/GM-CSF receptor family. Cell Signal 10:619-628.
Hayashida K, Kitamura T, Gorman D M, Arai K, Yokota T, Miyajima A (1990) Molecular cloning of a second subunit of the receptor for human granulocyte-macrophage colony-stimulating factor (GM-CSF): reconstitution of a high-affinity GM-CSF receptor. Proc Natl Acad Sci USA 87:9655-9659.
Hendlin D, Stapley E O, Jackson M, Wallick H, Miller A K, Wolf F J, Miller T W, Chaiet L, Kahan F M, Foltz E L, Woodruff H B, Mata J M, Hernandez S, Mochales S (1969) Phosphonomycin, a new antibiotic produced by strains of streptomyces. Science 166:122-123.
Holzheimer R G (2001) Management of secondary peritonitis. In: Surgical Treatment: Evidence-Based and Problem-Oriented. Holzheimer R G, Mannick J A, editors. Munich: Zuckschwerdt.
Karageorgopoulos D E, Wang R, Yu X H, Falagas M E (2012) Fosfomycin: evaluation of the published evidence on the emergence of antimicrobial resistance in Gram-negative pathogens. J Antimicrob Chemother 67:255-268.
Kaushansky K, O'Hara P J, Berkner K, Segal G M, Hagen F S, Adamson J W (1986) Genomic cloning, characterization, and multilineage growth-promoting activity of human granulocyte-macrophage colony-stimulating factor. Proc Natl Acad Sci USA 83:3101-3105.
Kitamura T, Hayashida K, Sakamaki K, Yokota T, Arai K, Miyajima A (1991) Reconstitution of functional receptors for human granulocyte/macrophage colony-stimulating factor (GM-CSF): evidence that the protein encoded by the AIC2B cDNA is a subunit of the murine GM-CSF receptor. Proc Natl Acad Sci USA 88:5082-5086.
Lanfrancone L, Boraschi D, Ghiara P, Falini B, Grignani F, Peri G, Mantovani A, Pelicci P G (1992) Human peritoneal mesothelial cells produce many cytokines (granulocyte colony-stimulating factor [CSF], granulocyte-monocyte-CSF, macrophage-CSF, interleukin-1 [IL-1], and IL-6) and are activated and stimulated to grow by IL-1. Blood 80:2835-2842.
Lopez A F, Vadas M A, Woodcock J M, Milton S E, Lewis A, Elliott M J, Gillis D, Ireland R, Olwell E, Park L S (1991) Interleukin-5, interleukin-3, and granulocyte-macrophage colony-stimulating factor cross-compete for binding to cell surface receptors on human eosinophils. J Biol Chem 266:24741-24747.
Moonen P, Mermod J J, Ernst J F, Hirschi M, DeLamarter J F (1987) Increased biological activity of deglycosylated recombinant human granulocyte/macrophage colony-stimulating factor produced by yeast or animal cells. Proc Natl Acad Sci USA 84:4428-4431.
Orozco H, Arch J, Medina-Franco H, Pantoja J P, Gonzalez Q H, Vilatoba M, Hinojosa C, Vargas-Vorackova F, Sifuentes-Osornio J (2006) Molgramostim (GM-CSF) associated with antibiotic treatment in nontraumatic abdominal sepsis: a randomized, double-blind, placebo-controlled clinical trial. Arch Surg 141:150-153.
Pachon-Ibanez M E, Ribes S, Dominguez M A, Fernandez R, Tubau F, Ariza J, Gudiol F, Cabellos C (2011) Efficacy of fosfomycin and its combination with linezolid, vancomycin and imipenem in an experimental peritonitis model caused by a Staphylococcus aureus strain with reduced susceptibility to vancomycin. Eur J Clin Microbiol Infect Dis 30:89-95.
Rodriguez A, Gallego A, Olay T, Mata J M (1977) Bacteriological evaluation of fosfomycin in clinical studies. Chemotherapy 23 Suppl 1:247-258.
Sato N, Sakamaki K, Terada N, Arai K, Miyajima A (1993) Signal transduction by the high-affinity GM-CSF receptor: two distinct cytoplasmic regions of the common beta subunit responsible for different signaling. EMBO J 12:4181-4189.
Schäfer K, Stöteknuel S, Schollmeyer P, Dobos G J (1998) Granulocyte macrophage-colony stimulating factor stimulates secretion of chemoattractive cytokines by peritoneal macrophages of CAPD patients. Adv Perit Dial 14:164-167.
Selgas R, Fernández de Castro M, Jimenez C, Cárcamo C, Contreras T, Bajo M A, Vara F, Corbí A (1996) Immunomodulation of peritoneal macrophages by granulocyte-macrophage colony-stimulating factor in humans. Kidney Int 50:2070-2078.
Shanafelt A B, Miyajima A, Kitamura T, Kastelein R A (1991a) The amino-terminal helix of GM-CSF and IL-5 governs high affinity binding to their receptors. EMBO J 10:4105-4112.
Shanafelt A B, Johnson K E, Kastelein R A (1991b) Identification of critical amino acid residues in human and mouse granulocyte-macrophage colony-stimulating factor and their involvement in species specificity. J Biol Chem 266:13804-13810.
Shinagawa N, Mizuno A, Mashita K, Yura J, Ishikawa S, Hirata K, Denno R, Mukaiya M, Ishibiki K, Aikawa N, et al (1994) [Bacteria isolated from intraabdominal infection and their susceptibilities to antimicrobial agents]. [Article in Japanese]. Jpn J Antibiot 47:1329-1343.
Shree N, Arora B S, Mohil R S, Kasana D, Biswal I (2013) Bacterial profile and patterns of antimicrobial drug resistance in intra-abdominal infections: Current experience in a teaching hospital. Indian J Pathol Microbiol 56:388-392.
Tobudic S, Matzneller P, Stoiser B, Wenisch J M, Zeitlinger M, Vychytil A, Jaeger W, Boehmdorfer M, Reznicek G, Burgmann H (2012) Pharmacokinetics of intraperitoneal and intravenous fosfomycin in automated peritoneal dialysis patients without peritonitis. Antimicrob Agents Chemother 56:3992-3995.
Toda H, Murata A, Oka Y, Uda K, Tanaka N, Ohashi I, Mori T, Matsuura N (1994) Effect of granulocyte-macrophage colony-stimulating factor on sepsis-induced organ injury in rats. Blood 83:2893-2898.
Troidle L, Finkelstein F (2006) Treatment and outcome of CPD-associated peritonitis. Ann Clin Microbiol Antimicrob 5:6.
Volk H D, Thieme M, Heym S, Döcke W D, Ruppe U, Tausch W, Manger D, Zuckermann S, Golosubow A, Nieter B, et al (1991) Alterations in function and phenotype of monocytes from patients with septic disease—predictive value and new therapeutic strategies. Behring Inst Mitt (88):208-215.
Wiest R, Krag A, Gerbes A (2012) Spontaneous bacterial peritonitis. Gut 61:297-310.
Wong G G, Witek J S, Temple P A, Wilkens K M, Leary A C, Luxenberg D P, Jones S S, Brown E L, Kay R M, Orr E C, et al (1985) Human GM-CSF: molecular cloning of the complementary DNA and purification of the natural and recombinant proteins. Science 228:810-815.

1. A method of treating or inhibiting a secondary infectious peritonitis or intra-abdominal infection of bacterial origin in a subject comprising:
administering to a subject having a secondary infectious peritonitis or intra-abdominal infection of bacterial origin by intraperitoneal administration a composition comprising a granulocyte-macrophage colony-stimulating factor (GM-CSF) in combination with fosfomycin, and a compound selected from the group consisting of metronidazole, ertapenem, imipenem and meropenem wherein the incidence of local peritonitis or intra-abdominal abscess in said subject is reduced.

2-20. (canceled)

21. The method of claim 1, wherein said subject has a postoperative perforated appendicitis.

22. The method of claim 1, wherein the composition comprises a dose of between 5 and 100 microgram of GM-CSF.

23. The method of claim 1, wherein the composition comprises a dose of between 1 gram and 12 gram of fosfomycin.

24. The method of claim 1, wherein the composition comprises a dose of between 250 milligram and 3 gram of metronidazole.

25. The method of claim 1, wherein the composition is administered in a volume of between 500 mL and 1500 mL.

26. The method of claim 1, wherein the composition has a pH of between 6.5 and 8.0.

27. The method of claim 1, wherein the composition comprises a dose of 50 microgram of GM-CSF, 4 gram of fosfomycin and 1 gram of metronidazole in a total volume of aqueous solution of 500 mL.

28. The method of claim 1, wherein the composition is administered between 1 and 6 times per day.

29. The method of claim 1, wherein the subject is a mammal.

30. The method of claim 29, wherein the subject is a human being.

31. The method of claim 30, wherein the human being is younger than 15 years of age.

32. The method of claim 30, wherein the human being is 15 years of age or older.

33. A kit comprising:
(i) a first composition comprising GM-CSF;
(ii) a second composition comprising a pharmaceutically acceptable salt of fosfomycin; and
(iii) a third composition comprising a compound selected from metronidazole, ertapenem, imipenem or meropenem,
wherein said first, second and third compositions are presented as solids or aqueous solutions formulated for intraperitoneal administration and are, optionally, mixed before such administration.

34. The kit of claim 33, wherein the third composition comprises metronidazole.

35. The kit of claim 33, wherein the kit comprises 50 microgram of GM-CSF, 4 gram of fosfomycin and 1 gram of metronidazole in a total volume of aqueous solution of 500 mL.

36. A method of treating or ameliorating secondary infectious peritonitis or intra-abdominal infection of bacterial origin in a subject having local peritonitis or intra-abdominal abscess comprising administering into a peritoneal cavity of said subject a composition comprising granulocyte-macrophage colony-stimulating factor (GM-CSF), in combination with fosfomycin and a compound selected from metronidazole, ertapenem, imipenem or meropenem.

37. The method according to claim 36, wherein the composition comprises metronidazole.

38. The method according to claim 36, wherein the composition comprises between 5 and 100 micrograms of GM-CSF, between 1 gram and 12 gram of fosfomycin and between 250 milligram and 3 gram of metronidazole.

39. The method according claim 36, wherein the composition comprises 50 microgram of GM-CSF, 4 gram of fosfomycin and 1 gram of metronidazole in a total volume of aqueous solution of 500 mL.
